(19) United States
(12) Patent Application Publication
(10) Pub. No.: US 2026/0108666 A1
(43) Pub. Date: Apr. 23, 2026

(54) ECMO CANNULA SYSTEMS AND METHODS

(71) Applicant: Cultiv8 Medical, LLC, Minneapolis, MN (US)

(72) Inventors: Stephen P. MICHAEL, Maple Grove, MN (US); Robert E. ATKINSON, Minneapolis, MN (US)

(73) Assignee: Cultiv8 Medical, LLC, Minneapolis, MN (US)

(21) Appl. No.: 19/363,962

(22) Filed: Oct. 21, 2025

Related U.S. Application Data
(60) Provisional application No. 63/710,187, filed on Oct. 22, 2024.

(51) Int. Cl. A61M 1/36; A61M 39/06

(52) U.S. Cl. CPC A61M 1/3659; A61M 39/06; A61M 2039/0626; A61M 2202/0021; A61M 2202/0413; A61M 2205/0216; A61M 2205/0222; A61M 2205/0238; A61M 2209/088; A61M 2210/04; A61M 2210/12

(57) ABSTRACT

A cannula system includes: a tubular cannula shaft including an inner liner defining a blood flow lumen, an outer layer, and a metallic support structure positioned therebetween, where the inner liner includes an anticoagulant coating on an inner surface, and the outer layer includes a lubricious coating on an outer surface; a boot including a skin securement element for securing the cannula system to skin of a patient and a stepped distal ledge configurable to mitigate insertion into a blood vessel after insertion of the tubular cannula shaft into the blood vessel, where the tubular cannula shaft distal of the boot has a constant diameter configurable for insertion into the blood vessel; a transition section defining a clamp region; and a hub assembly including a blood infusion port, a blood reperfusion port, and an intervention port, each of the ports in fluid communication with the blood flow lumen.

### CROSS-REFERENCE TO RELATED APPLICATIONS

This application claims priority to U.S. Provisional Application No. 63/710,187, filed Oct. 22, 2024, and is related to U.S. patent application Ser. No. 18/644,816, filed Apr. 24, 2024, and the entire disclosures of which are incorporated herein by reference.

### TECHNICAL FIELD

The present disclosure generally relates to cannula systems that are intended to be inserted into the vasculature to provide a fluid circuit for extracorporeal membrane oxygenation (ECMO).

### BACKGROUND

Currently available peripheral ECMO cannulas are generally designed to be inserted into the vasculature using a surgical approach. More recent advances in peripheral ECMO cannulas allow for percutaneous insertion, but often require some surgical cut-down.

### SUMMARY OF THE DISCLOSURE

In accordance with aspects of the disclosure, the present invention provides a cannula system comprising: a tubular cannula shaft having a distal end and a proximal end, the tubular cannula shaft including an inner liner formed of a lubricious material and defining a blood flow lumen therein, an outer layer, and a metallic support structure positioned between the inner liner and the outer layer, wherein the tubular cannula shaft includes blood perfusion holes at the distal end, the inner liner includes an anticoagulant coating on an inner surface thereof, and the outer layer includes a lubricious coating on an outer surface thereof; a boot having a distal end and a proximal end, the boot including at least one skin securement element for securing the cannula system to skin of a patient, wherein the boot is disposed about a portion of the tubular cannula shaft, the boot has a stepped distal ledge configurable to mitigate insertion into a blood vessel of the patient after insertion of the tubular cannula shaft into the blood vessel, the boot is longitudinally fixed relative to the tubular cannula shaft and rotatable relative to the tubular cannula shaft, and the tubular cannula shaft that is distal of the boot has a constant diameter configurable for insertion into the blood vessel; a transition section including a distal end portion and a proximal end portion, wherein the distal end portion has a diameter approximating the constant diameter of the tubular cannula shaft that is distal of the boot, the proximal end portion has a diameter larger than the constant diameter of the tubular cannula shaft that is distal of the boot, and the proximal end portion of the transition section extends proximally of the boot to define a clamp region; and a hub assembly connected to a proximal end of the clamp region, the hub assembly including a blood infusion port, a blood reperfusion port, and an intervention port, wherein each of the ports is in fluid communication with the blood flow lumen of the tubular cannula shaft.

The present invention further provides a method, comprising: providing a cannula system that comprises: a tubular cannula shaft having a distal end and a proximal end, the tubular cannula shaft including an inner liner formed of a lubricious material and defining a blood flow lumen therein, an outer layer, and a metallic support structure positioned between the inner liner and the outer layer, wherein the tubular cannula shaft includes blood perfusion holes at the distal end, the inner liner includes an anticoagulant coating on an inner surface thereof, and the outer layer includes a lubricious coating on an outer surface thereof; a boot having a distal end and a proximal end, the boot including at least one skin securement element for securing the cannula system to skin of a patient, wherein the boot is disposed about a portion of the tubular cannula shaft, the boot has a stepped distal ledge configurable to mitigate insertion into a blood vessel of the patient after insertion of the tubular cannula shaft into the blood vessel, the boot is longitudinally fixed relative to the tubular cannula shaft and rotatable relative to the tubular cannula shaft, and the tubular cannula shaft that is distal of the boot has a constant diameter configurable for insertion into the blood vessel; a transition section including a distal end portion and a proximal end portion, wherein the distal end portion has a diameter approximating the constant diameter of the tubular cannula shaft that is distal of the boot, the proximal end portion has a diameter larger than the constant diameter of the tubular cannula shaft that is distal of the boot, and the proximal end portion of the transition section extends proximally of the boot to define a clamp region; and a hub assembly connected to a proximal end of the clamp region, the hub assembly including a blood infusion port, a blood reperfusion port, and an intervention port, wherein each of the ports is in fluid communication with the blood flow lumen of the tubular cannula shaft.

The present invention further provides a method of using a cannula system comprising a tubular cannula shaft having a distal end and a proximal end, the tubular cannula shaft including an inner liner formed of a lubricious material and defining a blood flow lumen therein, an outer layer, and a metallic support structure positioned between the inner liner and the outer layer, wherein the tubular cannula shaft includes blood perfusion holes at the distal end, the inner liner includes an anticoagulant coating on an inner surface thereof, and the outer layer includes a lubricious coating on an outer surface thereof, a boot having a distal end and a proximal end, the boot including at least one skin securement element for securing the cannula system to skin of a patient, wherein the boot is disposed about a portion of the tubular cannula shaft, the boot has a stepped distal ledge configurable to mitigate insertion into a blood vessel of the patient after insertion of the tubular cannula shaft into the blood vessel, the boot is longitudinally fixed relative to the tubular cannula shaft and rotatable relative to the tubular cannula shaft, and the tubular cannula shaft that is distal of the boot has a constant diameter configurable for insertion into the blood vessel, a transition section including a distal end portion and a proximal end portion, wherein the distal end portion has a diameter approximating the constant diameter of the tubular cannula shaft that is distal of the boot, the proximal end portion has a diameter larger than the constant diameter of the tubular cannula shaft that is distal of the boot, and the proximal end portion of the transition section extends proximally of the boot to define a clamp region, and a hub assembly connected to a proximal end of the clamp region, the hub assembly including a blood infusion port, a blood reperfusion port, and an intervention port, wherein each of the ports is in fluid communication with the blood flow lumen of the tubular cannula shaft, the method comprising: inserting the tubular cannula shaft into the blood vessel of the patient, such that the stepped distal ledge of the boot is against a surface of the skin of the patient.

Thus, the disclosure may provide an ECMO cannula that allows for improved percutaneous insertion (without surgical cut-down) such that an interventionalist could quickly and safely insert the cannula into the vasculature. The embodiments described herein may address this unmet need, or other needs.

Such improvements are described in more detail hereinafter with reference to the drawings. The above summary is not intended to describe each and every embodiment or implementation of the present disclosure.

### BRIEF DESCRIPTION OF THE DRAWINGS

The drawings illustrate example embodiments of the present disclosure and, along with the description, serve to explain the principles of the disclosure. The drawings are only illustrative of certain embodiments and do not limit the disclosure or invention.

FIG. 1 is a perspective assembly view of an ECMO cannula system according to an embodiment of the disclosure.

FIG. 2 is a perspective assembly view of an alternative ECMO cannula system according to an embodiment of the disclosure.

FIG. 3 is a side assembly view of the ECMO cannula system shown in FIG. 2.

FIG. 3A is a detailed view taken at circle A in FIG. 2.

FIGS. 3B and 3C are detailed views taken at circles B and C, respectively, in FIG. 3.

FIG. 4 is an isometric view of the cannula of the system shown in FIG. 2.

FIG. 5 is an isometric view of the dilator of the system shown in FIG. 2.

FIG. 6 is a perspective assembly view of another alternative ECMO cannula system according to an embodiment of the disclosure.

FIG. 7 is a side schematic view of a vent accessory for use with the ECMO systems described herein.

While embodiments of the disclosure are amenable to various modifications and alternative forms, specifics thereof have been shown by way of example in the drawings and will be described in some detail. It should be understood, however, that the intention is not to limit the disclosure to the particular embodiments described. On the contrary, the intention is to cover all modifications, equivalents, and alternatives falling within the spirit and scope of the invention.

### DETAILED DESCRIPTION OF EMBODIMENTS

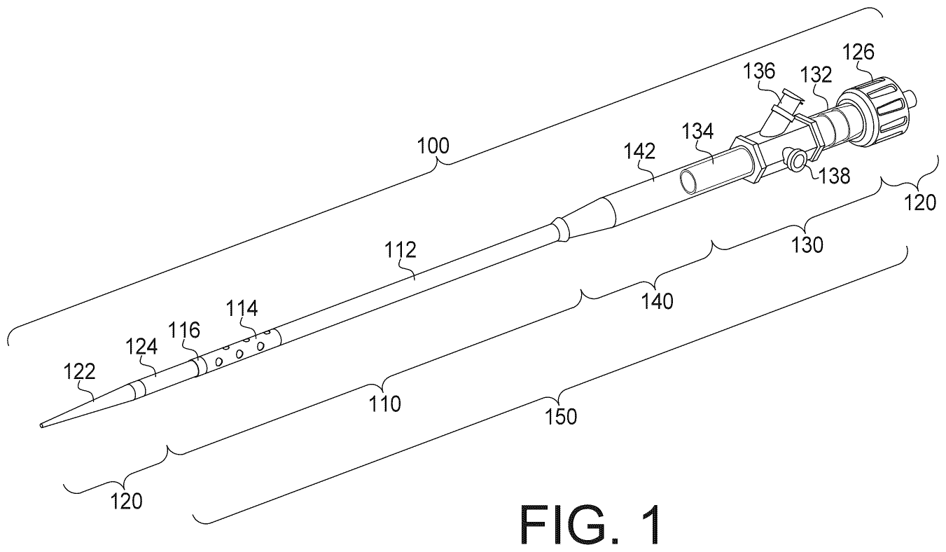

With reference to FIG. 1, an assembly view of an Extracorporeal Membrane Oxygenation (ECMO) cannula system (“cannula system”) 100 according to an embodiment of the present disclosure is shown. In general, the cannula system 100 may include a tubular cannula shaft 110, a transition section 140 connected to the proximal end of the tubular cannula shaft 110, a hub assembly 130 connected to a proximal end of the transition section 140, and a dilator assembly 120 extending therethrough. Collectively, the tubular cannula shaft 110, the transition section 140 and the hub assembly 130 may be referred to as the cannula 150, into which the dilator assembly 120 may be inserted. The cannula system 100 may be inserted into the vasculature as a system, with the transition section 140 and hub assembly 130 remaining outside the body. Once placed, the dilator assembly 120 may be removed to define a conduit through the hub assembly 130, transition section 140 and tubular cannula shaft 110 into the vasculature.

The tubular cannula shaft 110 may comprise, for example, a composite tubular shaft (“tubular shaft”) 112 including an inner layer or liner (e.g., at least one of polytetrafluoroethylene (PTFE) or high-density polyethylene (HDPE)) defining a blood flow lumen, a metallic support structure (e.g., a coil such as a stainless-steel coil) disposed about the inner layer, and an outer layer (e.g., polyether-block-amide) disposed over the metallic support structure. The outside of the tubular shaft 112 may be coated with a lubricious coating such as a hydrophilic coating to ease insertion. The inside of the tubular shaft 112, as well as all other inside surfaces, may be coated with an anticoagulant coating such as heparin. The proximal end of the tubular shaft 112 may be connected to the transition section 140 by thermal or adhesive bond, for example. The distal end of the tubular shaft 112 may include perfusion holes 114 formed by laser ablation, for example. To facilitate visualization via fluoroscopy, a radiopaque marker band may be incorporated into the distal tip 116 of the tubular shaft 112 between the layers thereof, or the polymer jacket may be loaded with radiopaque material (e.g., tungsten) at the distal tip 116, for example.

The transition section 140 may include a tapered elastomeric tube 142 as a clamping review, suitable for cross-clamping (to regulate blood flow) such as a clear polyurethane, for example. The clamping region may be configured to deform when a clamp is clamped thereto and closed, to stop blood flow in the blood flow lumen. The transition section 140 may have a constant diameter portion for cross-clamping and a distal cone section to transition down to the diameter of the tubular shaft 112. The transition section 140 may be connected to the hub assembly 130 via a compression fit onto a barbed section 134, for example. The transition section 140 may include a distal end portion and a proximal end portion, wherein the distal end portion has a diameter approximating the constant diameter of the tubular cannula shaft 110 that is distal of the boot 224, and the proximal end portion may have a diameter larger than the constant diameter of the tubular cannula shaft 110 that is distal of the boot 224.

The dilator assembly 120 may comprise a relatively rigid LDPE tube, for example, with a tapered distal end 122, a main body portion 124 having a constant diameter, and a hub assembly including a proximal hub 126 that interlocks with the hub assembly 130. The lumen of the dilator assembly 120 extends the entire length thereof to accommodate a guide wire (e.g., 0.035″ guidewire compatible, not shown), over which the cannula system 100 is delivered. The main body portion 124 may have an outer diameter that closely matches the inside diameter of the distal tip 116, with an interference fit or a very small gap to allow relative movement therebetween. The tapered distal end 122 of the dilator provides gradual dilatation of the vascular puncture site and a smooth transition from the guidewire to the distal tip 116. To facilitate visualization via fluoroscopy, the dilator assembly 120 may be loaded with a radiopaque material such as barium sulfate. The proximal hub 126 may include an interlocking feature that mates with, and releasably secures to, the hub assembly 130. For example, the proximal hub 126 may include a polymer plug that seats in the inside of the proximal barbed section 132.

The hub assembly 130 is generally hollow defining a common space or lumen therethrough and may be formed an injection molded polymer, for example. The hub assembly 130 may include the barbed section 134 for connection the transition section 140, a proximal barbed section 132 for connection to ECMO tubing, an angled side port 136 (e.g., an intervention portion), which optionally may include a Touhy Borst hemostatic valve for the introduction of an ancillary device such as other intravascular devices, and a reperfusion side port 138 for connection to tubing that is connect to an infusion cannula inserted into the distal limb (e.g., femoral artery) for purposes of distal reperfusion. In general, providing the angled side port 136 may allow ancillary devices such as guidewires, diagnostic catheters, balloon catheters, stent delivery catheters, etc. to be introduced via the same cannula system without requiring an additional vascular access site with the associated puncture and closure issues. Each of the angled side port 136 and the reperfusion port 138, as well as a perfusion port on an end of the cannula system 100, may be in fluid communication with the blood flow lumen of the tubular cannula shaft 110.

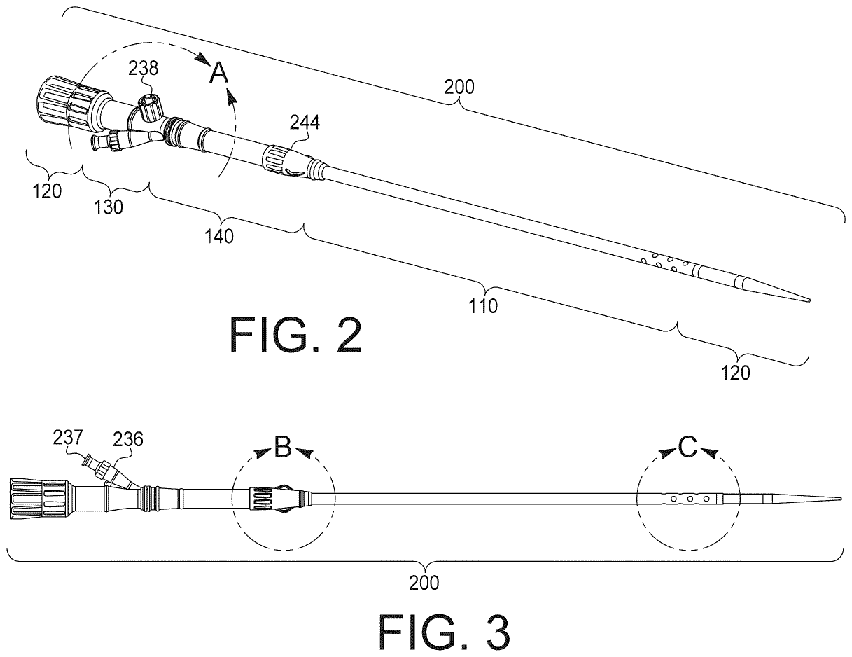

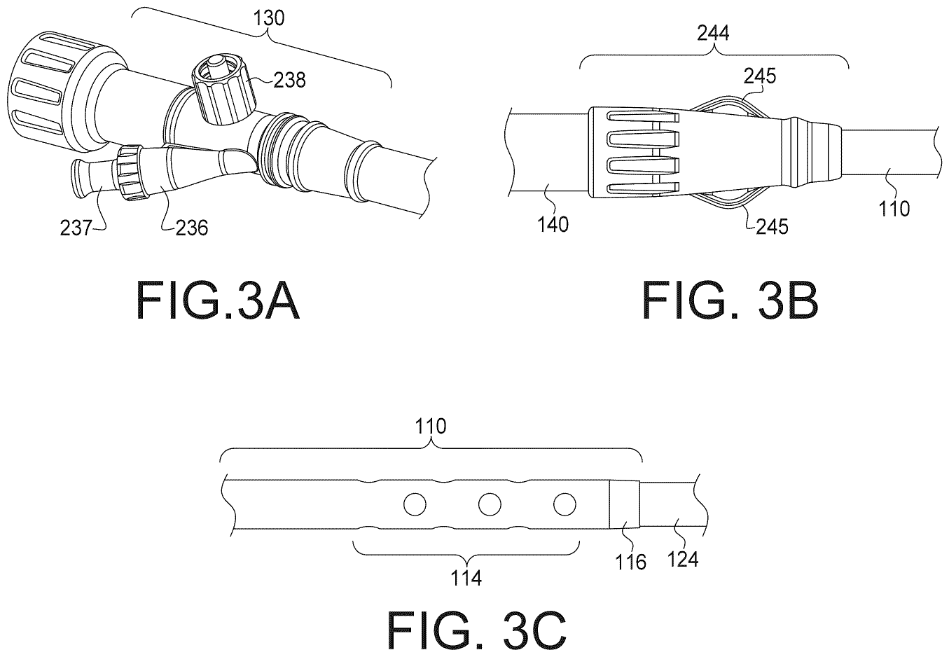

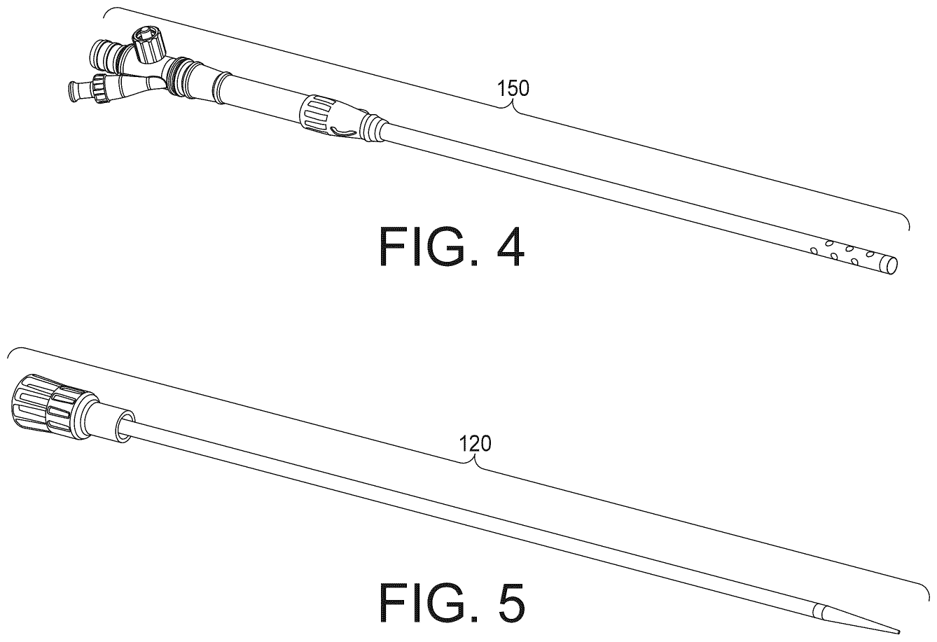

With reference to FIGS. 2 and 3, isometric and side views of a cannula system 200 are shown. The cannula system 200 may be the same or similar in terms of construction, function and use as cannula system 100, except as otherwise shown and described herein, wherein similar components and features are numbered the same. As in the prior embodiment, the cannula system 200 may include a cannula 150 (including hub assembly 130, transition section 140 and tubular cannula shaft 110) and a dilator assembly 120, which are shown disassembled in FIGS. 4 and 5, respectively. In some embodiments, a material of the tubular cannula shaft 110 has a higher durometer than a durometer of a material of the transition section 140. In some examples, the material of the tubular cannula shaft 110 includes a thermopolymer. In some examples, a material of the clamp region includes an elastomer. In some examples, the thermopolymer includes a nylon, or a polyether-block-amide. In some examples, the thermopolymer has a durometer of 40D or more, of 50D or more, of 60D or more, or of 70D or more. In some examples, the elastomer has a durometer of 40A or less, or of 30A or less.

In an embodiment, the perfusion side port 238 may be different than the reperfusion side port 138 described previously. The perfusion side port 238 is generally shown in FIG. 2 and shown in detail in FIG. 3A. The perfusion side port 238 may comprise a male Luer fitting for easier connection to conventional arterial line that has a female Luer fitting. Such arterial lines may be used for distal perfusion of the lower limb downstream of the femoral access site, which can otherwise have obstructed blood flow due to the presence of the ECMO cannula therein.

Also in an embodiment, a boot 244 may be provided over and around the connection between the transition section 140 and the tubular cannula shaft 110. The boot 244 is generally shown in FIGS. 2 and 3 and shown in detail in FIG. 3B. The boot 244 may provide for strain relief at the junction between the transition section 140 and the tubular cannula shaft 110. The boot 244 may also provide means to anchor the cannula 150 to the patient's leg using skin securement elements 245, in the form of, e.g., two suture loops defining opening to receive sutures, for example. The boot 244 may include an internal circumferential spline to allow for relative rotational movement of the tubular cannula shaft 110 and the hub assembly 130 while limiting or preventing relative longitudinal movement thereof.

Further in an embodiment, an angled side port 236 may be different than the angled side port 136 shown and described previously. The angled side port 236 is generally shown in FIGS. 2 and 3 and shown in detail in FIG. 3A. The angled side port 236 may include a hemostatic valve disposed therein to seal around interventional devices inserted therethrough. The angled side port 236 may include a removable plug 237. The removable plug 237 may occupy the dead space inside the angled side port 236 while not in use to mitigate stagnant blood flow and thrombus formation.

Further yet in an embodiment, the tubular cannula shaft 110 may be formed differently. With reference to FIG. 3C, at the portion of the tubular cannula shaft 110 where the perfusion holes 114 are located, the metallic support structure embedded between layers may be replaced with a stainless steel hypotube with holes formed therein, for example. This configuration avoids forming holes in the coiled layer to mitigate coil turns from becoming dislodged. The construction may thus comprise, for example, an inner liner of PTFE with a 0.240″ ID and 0.003″ wall thickness, an overlay coil (e.g., metallic support structure) of 0.005″ by 0.002″ stainless steel ribbon wound at a 0.030″ pitch extending through the tubular shaft 112, a 5 cm long 0.002″ wall thickness stainless steel hypotube with holes and optional spiral cuts extending from and optionally interlocked with the distal end of the coil and extending through the section with perfusion holes 114 (12 holes over 5 cm in 4 quadrants), a jacket layer of 40D Pebax (natural/clear color for blood and bubble visibility) and 0.010″ wall thickness overlaying the coil and hypotube, and a radiopaque loaded polymer thermally bonded at the distal tip thereof.

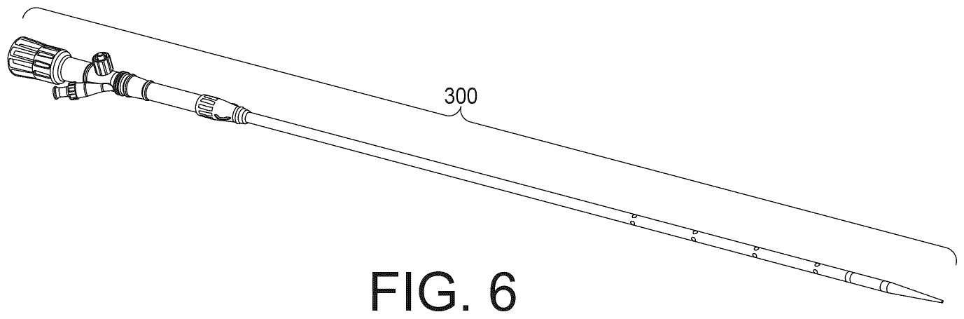

With reference to FIG. 6, a cannula system 300 may be a longer version of the ECMO cannula systems previously shown and described, and may be a venous cannula system. Venous cannulas are generally longer and have more perfusion holes than arterial cannulas. The cannula systems 100 and 200 may be suitable for arterial applications, whereas cannula system 300 may be suitable for venous applications. In general, the cannula systems 100 and 200 may extend about 15 cm to 25 cm into the arterial system with 12 perfusion holes over 5 cm, for example, and the cannula system 300 may extend about 50 cm to 60 cm into the venous system with 16 to 20 perfusion holes over 15 cm, for example.

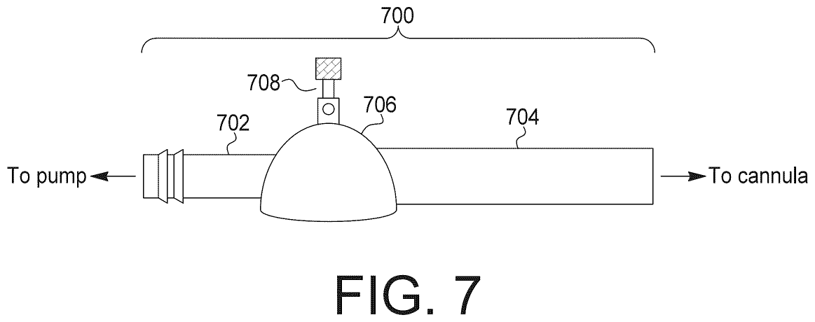

With reference to FIG. 7, a bubble capture and vent accessory 700 is shown schematically. The accessory 700 may be a separate part from the cannula system that is operably connectable thereto, or it may be functionally incorporated into the hub of the cannula system. The accessory 700 may be used to facilitate connection from the ECMO tubing (typically ⅜″ ID, ½″ OD Tygon) to the cannula hub. For connection to the Tygon tubing leading to the ECMO machine, a ⅜″ barbed fitting 702 may be provided. For connection to the ECMO cannula, a tube 704 with a ⅜″ inner diameter may be provided. Between the barbed fitting 702 and the tube 704, a transparent, dome-shaped bubble chamber 706 may be provided to collect any air bubbles that are introduced during connection. The dome shape encourages air bubbles to rise to the vertical apex where a vent 708 is disposed. The vent 708 may be opened to allow air to escape and subsequently closed to mitigate the ingress of air.

The disclosure may include methods associated with the cannula system shown and described. For example, the disclosure may include a method, which includes providing the cannula system. A method of using the cannular system may include, for example, one or more of the following: inserting the tubular cannula shaft into the blood vessel of the patient, such that the stepped distal ledge of the boot is against a surface of the skin of the patient; securing the cannula system to the skin of the patient with the at least one skin securement element; suturing the cannula system to the skin of the patient with a suture that extends through the at least one skin securement element and the skin of the patient; inserting an ancillary device through the intervention port, such that the ancillary device extends into the blood flow lumen; flowing blood through a tube connected to the blood reperfusion port to another blood vessel of the patient; or flowing blood through a tube connected to the blood infusion port into the blood flow lumen and subsequently into the blood vessel of the patient or any other step.

All of the aspects described in the present disclosure (including references incorporated by reference, accompanying claims, abstract and drawings), may be combined in any order, in part or in full, or in any combination or modification, except when such are incompatible or inconsistent. Furthermore, each aspect may be replaced by alternative features serving the same, equivalent or similar purpose, unless expressly stated otherwise or inconsistent with the teachings herein. Thus, unless expressly stated otherwise, each aspect disclosed herein may be only an example of equivalent or similar features. It is intended that the invention be defined by the attached claims and their legal equivalents.

1. A cannula system comprising:
a tubular cannula shaft having a distal end and a proximal end, the tubular cannula shaft including an inner liner formed of a lubricious material and defining a blood flow lumen therein, an outer layer, and a metallic support structure positioned between the inner liner and the outer layer, wherein the tubular cannula shaft includes blood perfusion holes at the distal end, the inner liner includes an anticoagulant coating on an inner surface thereof, and the outer layer includes a lubricious coating on an outer surface thereof;
a boot having a distal end and a proximal end, the boot including at least one skin securement element for securing the cannula system to skin of a patient, wherein the boot is disposed about a portion of the tubular cannula shaft, the boot has a stepped distal ledge configurable to mitigate insertion into a blood vessel of the patient after insertion of the tubular cannula shaft into the blood vessel, the boot is longitudinally fixed relative to the tubular cannula shaft and rotatable relative to the tubular cannula shaft, and the tubular cannula shaft that is distal of the boot has a constant diameter configurable for insertion into the blood vessel;
a transition section including a distal end portion and a proximal end portion, wherein the distal end portion has a diameter approximating the constant diameter of the tubular cannula shaft that is distal of the boot, the proximal end portion has a diameter larger than the constant diameter of the tubular cannula shaft that is distal of the boot, and the proximal end portion of the transition section extends proximally of the boot to define a clamp region; and
a hub assembly connected to a proximal end of the clamp region, the hub assembly including a blood infusion port, a blood reperfusion port, and an intervention port, wherein each of the ports is in fluid communication with the blood flow lumen of the tubular cannula shaft.

2. The cannula system of claim 1, wherein a material of the tubular cannula shaft has a higher durometer than a material of the transition section.

3. The cannula system of claim 1, wherein a material of the tubular cannula shaft includes a thermopolymer, and a material of the clamp region includes an elastomer, the thermopolymer includes at least one of a nylon or a polyether-block-amide, the thermopolymer has a durometer of 40D or more, and the elastomer has a durometer of 40A or less.

4. The cannula system of claim 1, wherein the lubricious material of the inner liner includes at least one of polytetrafluoroethylene (PTFE) or high-density polyethylene (HDPE), and the coating of the outer layer is hydrophilic.

5. The cannula system of claim 1, wherein the clamp region is configurable to receive a clamp that, when closed, deforms the clamp region to stop blood flow in the blood flow lumen.

6. The cannula system of claim 1, wherein the at least one skin securement element includes two skin securement elements configurable to receive sutures to secure the cannula system to the skin of the patient.

7. The cannula system of claim 1, wherein the intervention port is configurable to receive an ancillary device for introduction into the blood flow lumen, and intervention port includes a hemostatic valve configurable to seal around an ancillary device inserted into the intervention port.

8. The cannula system of claim 1, wherein the metallic support structure is in the form of a coil.

9. The cannula system of claim 1, wherein the boot is configurable to provide strain relief between the tubular cannula shaft and the transition section.

10. A method, comprising:
providing a cannula system that comprises:
a tubular cannula shaft having a distal end and a proximal end, the tubular cannula shaft including an inner liner formed of a lubricious material and defining a blood flow lumen therein, an outer layer, and a metallic support structure positioned between the inner liner and the outer layer, wherein the tubular cannula shaft includes blood perfusion holes at the distal end, the inner liner includes an anticoagulant coating on an inner surface thereof, and the outer layer includes a lubricious coating on an outer surface thereof;
a boot having a distal end and a proximal end, the boot including at least one skin securement element for securing the cannula system to skin of a patient, wherein the boot is disposed about a portion of the tubular cannula shaft, the boot has a stepped distal ledge configurable to mitigate insertion into a blood vessel of the patient after insertion of the tubular cannula shaft into the blood vessel, the boot is longitudinally fixed relative to the tubular cannula shaft and rotatable relative to the tubular cannula shaft, and the tubular cannula shaft that is distal of the boot has a constant diameter configurable for insertion into the blood vessel;
a transition section including a distal end portion and a proximal end portion, wherein the distal end portion has a diameter approximating the constant diameter of the tubular cannula shaft that is distal of the boot, the proximal end portion has a diameter larger than the constant diameter of the tubular cannula shaft that is distal of the boot, and the proximal end portion of the transition section extends proximally of the boot to define a clamp region; and
a hub assembly connected to a proximal end of the clamp region, the hub assembly including a blood infusion port, a blood reperfusion port, and an intervention port, wherein each of the ports is in fluid communication with the blood flow lumen of the tubular cannula shaft.

11. The method of claim 10, wherein a material of the tubular cannula shaft has a durometer of at least 40D, and a material of the clamp region has a durometer of at most 40A.

12. The method of claim 10, wherein the clamp region is configurable to receive a clamp that, when closed, deforms the clamp region to stop blood flow in the blood flow lumen.

13. The method of claim 10, wherein the intervention port includes a seal configurable to seal around an ancillary device inserted into the intervention port.

14. The method of claim 10, wherein the at least one skin securement element includes an opening configurable to receive a suture to secure the cannula system to the skin of the patient.

15. A method of using a cannula system comprising a tubular cannula shaft having a distal end and a proximal end, the tubular cannula shaft including an inner liner formed of a lubricious material and defining a blood flow lumen therein, an outer layer, and a metallic support structure positioned between the inner liner and the outer layer, wherein the tubular cannula shaft includes blood perfusion holes at the distal end, the inner liner includes an anticoagulant coating on an inner surface thereof, and the outer layer includes a lubricious coating on an outer surface thereof, a boot having a distal end and a proximal end, the boot including at least one skin securement element for securing the cannula system to skin of a patient, wherein the boot is disposed about a portion of the tubular cannula shaft, the boot has a stepped distal ledge configurable to mitigate insertion into a blood vessel of the patient after insertion of the tubular cannula shaft into the blood vessel, the boot is longitudinally fixed relative to the tubular cannula shaft and rotatable relative to the tubular cannula shaft, and the tubular cannula shaft that is distal of the boot has a constant diameter configurable for insertion into the blood vessel, a transition section including a distal end portion and a proximal end portion, wherein the distal end portion has a diameter approximating the constant diameter of the tubular cannula shaft that is distal of the boot, the proximal end portion has a diameter larger than the constant diameter of the tubular cannula shaft that is distal of the boot, and the proximal end portion of the transition section extends proximally of the boot to define a clamp region, and a hub assembly connected to a proximal end of the clamp region, the hub assembly including a blood infusion port, a blood reperfusion port, and an intervention port, wherein each of the ports is in fluid communication with the blood flow lumen of the tubular cannula shaft, the method comprising:
inserting the tubular cannula shaft into the blood vessel of the patient, such that the stepped distal ledge of the boot is against a surface of the skin of the patient.

16. The method of claim 15, further comprising:
securing the cannula system to the skin of the patient with the at least one skin securement element.

17. The method of claim 15, suturing the cannula system to the skin of the patient with a suture that extends through the at least one skin securement element and the skin of the patient.

18. The method of claim 15, further comprising:
inserting an ancillary device through the intervention port, such that the ancillary device extends into the blood flow lumen.

19. The method of claim 15, further comprising:
flowing blood through a tube connected to the blood reperfusion port to another blood vessel of the patient.

20. The method of claim 15, further comprising:
flowing blood through a tube connected to the blood infusion port into the blood flow lumen and subsequently into the blood vessel of the patient.
